(19) United States
(12) Patent Application Publication
(10) Pub. No.: US 2009/0004113 A1
(43) Pub. Date: Jan. 1, 2009

(54) Macrophage-Enhanced MRI (MEMRI)

(75) Inventor: Gerald L. Wolf, Westborough, MA (US)

Correspondence Address: BROMBERG & SUNSTEIN LLP, 125 SUMMER STREET, BOSTON, MA, 02110-1618, US

(73) Assignee: AMAG PHARMACEUTICALS, INC., Cambridge, MA (US)

(21) Appl. No.: 12/145,565

(22) Filed: Jun. 25, 2008

Related U.S. Application Data
(60) Provisional application No. 60/947,259, filed on Jun. 29, 2007.

(51) Int. Cl. A61K 49/06; A61K 49/00; A61B 5/055

(52) U.S. Cl. 424/9.2; 424/9.3; 600/420

(57) ABSTRACT

Methods for assessing stage of cancer in a subject are provided, comprising administering a macrophage imaging agent to the subject, making a magnetic resonance image of regions of the subject's body at cancer risk, and using the image to assess macrophage density and displacement associated with any primary cancer or metastatic cancer in the subject, such density and displacement being indicative of neoplasia. The macrophage imaging agent may be an ultrasmall superparamagnetic iron oxide particle and in particular embodiments, the macrophage imaging agent has a blood half-life sufficient to permit microphage trapping throughout the regions at cancer risk. Additional embodiments provide methods for assessing efficacy of an anticancer treatment in a subject, methods for determining frequency of follow-up MEMRI evaluation in a subject, methods for determining metastatic potential of cancer foci in a subject, and methods for determining prognosis of cancer in a subject. Methods for directing site of biopsy in a subject by performing a whole body MEMRI evaluation of the subject to identify macrophage density at a tumor site of interest and assessing the macrophage density to identify the site of biopsy in the subject, macrophage density being an indicator of tumor growth are also provided, in addition to methods for providing individualized cancer treatment to a subject in need thereof using whole body MEMRI evaluation.

### CROSS REFERENCE TO RELATED APPLICATIONS

The present application claims priority from provisional patent application U.S. Application Ser. No. 60/947,259, filed Jun. 29, 2007, which is hereby incorporated by reference herein.

### TECHNICAL FIELD

The present invention relates to whole body MRI scanning and cancer staging using macrophage-seeking MRI agents to perform Macrophage-Enhanced MRI or “MEMRI”.

### BACKGROUND ART

Cancer is one of the leading causes of death in the developed world, resulting in over 500,000 deaths per year in the United States alone. Over one million people are diagnosed with cancer in the U.S. each year, and overall it is estimated that more than 1 in 3 people will develop some form of cancer during their lifetime. Though there are more than 200 different types of cancer, four of them—breast, lung, colorectal, and prostate—account for over half of all new cases (Jemal et al., CA Cancer J. Clin. 53:5-26 (2003)). Cancer metastasis is considered to be due to the distribution of cancer cells via the blood—with liver, lung, bone, and CNS as common sites at risk, or the lymphatics with lymph node and bone as metastatic risk sites.

Breast cancer is the most common cancer in women, with an estimate 12% of women at risk of developing the disease during their lifetime. Although mortality rates have decreased due to earlier detection and improved treatments, breast cancer remains a leading cause of death in middle-aged women. Furthermore, metastatic breast cancer is still an incurable disease. On presentation, most patients with metastatic breast cancer have only one or two organ systems affected, but as the disease progresses, multiple sites usually become involved. The most common sites of metastatic involvement are locoregional recurrences in the skin and soft tissues of the chest wall, as well as in regional lymph nodes. The most common site for distant metastasis is the bone (30-40% of distant metastasis), followed by the lungs and liver. And although only approximately 1-5% of women with newly diagnosed breast cancer have distant metastasis at the time of diagnosis, approximately 50% of patients with local disease eventually relapse with metastasis within five years. At present the median survival from the manifestation of distant metastases is about three years.

Current methods of diagnosing and staging breast cancer include the tumor-node-metastasis (TNM) system that relies on tumor size, tumor presence in lymph nodes, and the presence of distant metastases as described in the American Joint Committee on Cancer, AJCC Cancer Staging Manual, Philadelphia, Pa., Lippincott-Raven Publishers, 6th ed. (2006), pp 221-240, and in Harris, J R: “Staging of breast carcinoma” in Harris, J. R., et al., eds., Breast Diseases, Philadelphia, Lippincott (1991). These parameters are used to provide a prognosis and select an appropriate therapy. The morphologic appearance of the tumor can also be assessed but because tumors with similar histopathologic appearance can exhibit significant clinical variability, this approach has serious limitations. Finally assays for cell surface markers can be used to divide certain tumors types into subclasses. For example, one factor considered in the prognosis and treatment of breast cancer is the presence of the estrogen receptor (ER) as ER-positive breast cancers typically respond more readily to hormonal therapies such as tamoxifen or aromatase inhibitors than ER-negative tumors. Yet these analyses, though useful, are only partially predictive of the clinical behavior of breast tumors, and there is much phenotypic diversity present in breast cancers that current diagnostic tools fail to detect and current therapies fail to treat.

Prostate cancer is the most common cancer in men in the developed world, representing an estimated 33% of all new cancer cases in the U.S., and is the second most frequent cause of death (Jemal et al., CA Cancer J. Clin. 53:5-26 (2003)). Since the introduction of the prostate specific antigen (PSA) blood test, early detection of prostate cancer has dramatically improved survival rates, and the five year survival rate for patients with local and regional stage prostate cancers at the time of diagnosis is nearing 100%. Yet more than 50% of patients will eventually develop locally advanced or metastatic disease (Muthuramalingam et al., Clin. Oncol. 16:505-516 (2004)).

Currently radical prostatectomy and radiation therapy provide curative treatment for the majority of localized prostate tumors. However, therapeutic options are very limited for advanced cases. For metastatic disease, androgen ablation with luteinizing hormone-releasing hormone (LHRH) agonist alone or in combination with anti-androgens is the standard treatment. Yet despite maximal androgen blockage, the disease nearly always progresses with the majority developing androgen-independent disease. At present there is no uniformly accepted treatment for hormone refractory prostate cancer, and chemotherapeutic regimes are commonly used (Muthuramalingam et al., Clin. Oncol. 16:505-516 (2004); Trojan et al., Anticancer Res. 25:551-561 (2005)).

Colorectal cancer is the third most common cancer and the fourth most frequent cause of cancer deaths worldwide (Weitz et al., 2005, Lancet 365:153-65). Approximately 5-10% of all colorectal cancers are hereditary with one of the main forms being familial adenomatous polyposis (FAP), an autosomal dominant disease in which about 80% of affected individuals contain a germline mutation in the adenomatous polyposis coli (APC) gene. Colorectal carcinoma has a tendency to invade locally by circumferential growth and elsewhere by lymphatic, hematogenous, transperitoneal, and perineural spread. The most common site of extralymphatic involvement is the liver, with the lungs the most frequently affected extra-abdominal organ. Other sites of hematogenous spread include the bones, kidneys, adrenal glands, and brain.

The current staging system for colorectal cancer is based on the degree of tumor penetration through the bowel wall and the presence or absence of nodal involvement. This staging system is defined by three major Duke's classifications: Duke's A disease is confined to submucosa layers of colon or rectum; Duke's B disease has tumors that invade through the muscularis propria and may penetrate the wall of the colon or rectum; and Duke's C disease includes any degree of bowel wall invasion with regional lymph node metastasis. While surgical resection is highly effective for early stage colorectal cancers, providing cure rates of 95% in Duke's A patients, the rate is reduced to 75% in Duke's B patients and the presence of positive lymph node in Duke's C disease predicts a 60% likelihood of recurrence within five years. Treatment of Duke's C patients with a post surgical course of chemotherapy reduces the recurrence rate to 40%-50%, and is now the standard of care for these patients.

Lung cancer is the most common cancer worldwide, the third most commonly diagnosed cancer in the United States, and by far the most frequent cause of cancer deaths (Spiro et al., Am. J. Respir. Crit. Care Med. 166:1166-1196 (2002); Jemal et al., CA Cancer J. Clin. 53:5-26 (2003)). Cigarette smoking is believed responsible for an estimated 87% of all lung cancers making it the most deadly preventable disease. Lung cancer is divided into two major types that account for over 90% of all lung cancers: small cell lung cancer (SCLC) and non-small cell lung cancer (NSCLC). SCLC accounts for 15-20% of cases and is characterized by its origin in large central airways and histological composition of sheets of small cells with little cytoplasm. SCLC is more aggressive than NSCLC, growing rapidly and metastasizing early and often. NSCLC accounts for 80-85% of all cases and is further divided into three major subtypes based on histology: adenocarcinoma, squamous cell carcinoma (epidermoid carcinoma), and large cell undifferentiated carcinoma. The most common metastatic sites are pleura, lung, bone, liver, brain, and pericardium.

Lung cancer typically presents late in its course, and thus has a median survival of only 6-12 months after diagnosis and an overall 5 year survival rate of only 5-10%. Although surgery offers the best chance of a cure, only a small fraction of lung cancer patients are eligible with the majority relying on chemotherapy and radiotherapy. Despite attempts to manipulate the timing and dose intensity of these therapies, survival rates have increased little over the last 15 years (Spiro et al., Am. J. Respir. Crit. Care Med. 166:1166-1196 (2002)).

Cancer arises from dysregulation of the mechanisms that control normal tissue development and maintenance, and increasingly stem cells are thought to play a central role (Beachy et al., Nature 432:324 (2004)). During normal animal development, cells of most or all tissues are derived from normal precursors, called stem cells (Morrison et al., Cell 88:287-298 (1997); Morrison et al., Curr. Opin. Immunol. 9:216-221 (1997); Morrison et al., Annu. Rev. Cell. Dev. Biol. 11:35-71 (1995)). Stem cells are cells that: (1) have extensive proliferative capacity; (2) are capable of asymmetric cell division to generate one or more kinds of progeny with reduced proliferative and/or developmental potential; and (3) are capable of symmetric cell divisions for self-renewal or self-maintenance.

Solid tumors are composed of heterogeneous cell populations. For example, breast cancers are a mixture of cancer cells and normal cells, including mesenchymal (stromal) cells, inflammatory cells, and endothelial cells. Classic models of cancer hold that phenotypically distinct cancer cell populations all have the capacity to proliferate and give rise to a new tumor. In the classical model, tumor cell heterogeneity results from environmental factors as well as ongoing mutations within cancer cells resulting in a diverse population of tumorigenic cells. This model rests on the idea that all populations of tumor cells would have some degree of tumorigenic potential. (Pandis et al., Genes, Chromosomes & Cancer 12:122-129 (1998); Kuukasjrvi et al., Cancer Res. 57:1597-1604 (1997); Bonsing et al., Cancer 71:382-391 (1993); Bonsing et al., Genes Chromosomes & Cancer 82:173-183 (2000); Beerman H. et al., Cytometry. 12:147-154 (1991); Aubele M & Werner M, Analyt. Cell. Path. 19:53 (1999); Shen L et al., Cancer Res. 60:3884 (2000)).

An alternative model for the observed solid tumor cell heterogeneity is that solid tumors result from a “solid tumor stem cell” that subsequently undergoes chaotic development through both symmetric and asymmetric rounds of cell divisions. In this stem cell model, solid tumors contain a distinct and limited (possibly even rare) subset of cells that share the properties of normal “stem cells”, in that they extensively proliferate and efficiently give rise both to additional solid tumor stem cells (self-renewal) and to the majority of tumor cells of a solid tumor that lack tumorigenic potential. Indeed, mutations within a long-lived stem cell population may initiate the formation of cancer stem cells that underlie the growth and maintenance of tumors and whose presence contributes to the failure of current therapeutic approaches.

MRI is noninvasive, tomographic, nonionizing, and able to generate images with high resolution and excellent soft tissue contrast. Whole body MRI has recently been used for evaluation of metastasis in bone in the absence of contrast agents. When MRI has been used in tumor staging, it has been by taking advantage of inherent tissue differences in MR properties that could be imaged by varying the MR image sequences. To obtain whatever information is contained in these inherent tissue differences for all tissues at cancer risk has required selection of different imaging sequences for each potential host tissue as well as the repeated programming and positioning of the patient within the MR instrument. It is known that the administration of some agents can change tissue MR properties in a useful way. The most popular agents for assessing the primary tumor are small gadolinium chelates. Given intravenously, these agents are distributed by blood perfusion and can identify regions of excess vascular leakiness or enlarged extracellular spaces that may herald the presence of cancer. However, these agents have no cell targeting capabilities and their distribution and accumulation are not specific for cancer nor for the tissue at risk for cancer metastasis. Bolus administration and dynamic MRI may provide some additional information about the degree of vascular leakiness, but such information can only be obtained for one body region of interest. As is described in example 1 (vide infra), the use of such contrast-enhanced MRI may be insufficient to characterize the cancer stage even with respect to the primary tumor.

Positive contrast agents cause a reduction in the T1 relaxation time (increased signal intensity on T1 weighted images). They are typically small molecular weight compounds containing as their active element Gadolinium, Manganese, or Iron. All of these elements have unpaired electron spins in their outer shells and long relaxivities.

Negative contrast agents (appearing predominantly dark on MRI) are small particulate aggregates often termed superparamagnetic iron oxides (SPIO) or ultrasmall superparamagnetic iron oxides (USPIO). USPIO typically are less than about 100 nanometers in diameter, and often have a mean diameter of less than 50 nm. These agents produce predominantly spin relaxation effects (local field inhomogeneities), which results in shorter T1 and T2 relaxation times. SPIO's and ultrasmall superparamagnetic iron oxides (USPIO) usually consist of a crystalline iron oxide core containing thousands of iron atoms and a shell of polymer, dextran, polyethyleneglycol, and produce very high T2 relaxivities.

For the lymph, liver and spleen images, ferumoxtran-10 is useful because it identifies normal healthy tissues that are enriched with macrophages. The reduced population of macrophage in the tumor in these particular types of tissue permits visualization of the tumor by the absence of enhancement relative to the normal tissue. In contrast, for brain tumors, it has been proposed that ferumoxtran-10 is useful to image brain tumors because of its association with reactive cells surrounding the tumor, including astrocytes and dendrites, which are found only in the brain and other nerve tissue and macrophages. This study (Nixon et al. Neuropath and Appl. Neurobiol (2004), 30, 456-471) focused on primary tumors in the brain, and the authors report that there was a pattern of sharply delimited cells without processes that were histologically identifiable as macrophages and another pattern of stellate-shaped cells typical of reactive astrocytes. This lead the authors to conclude that uptake in primary brain tumors of the iron oxide contrast agent studied is primarily concentrated in reactive cells in and around the brain tumor, rather than the tumor cells themselves. Thus, they could not conclude that all of the lesions imaged with the iron oxide contrast agent were actually tumors (id., at 464).

A special group of negative contrast agents (appearing dark on MRI) are perfluorocarbons (perfluorochemicals), because their presence excludes the hydrogen atoms responsible for the signal in MR imaging. These agents are reported to allow enhanced, sensitive detection and quantification of occult microthrombi within the intimal surface of atherosclerotic vessels in symptomatic patients and provide direct evidence to support acute therapeutic intervention, particularly if used in combination with gadolinium (see Flacke et al. Circulation. 2001; 104:1280).

In sum, the prior art teaches the use of contrast agents that are not specific to cancer at all, namely gadolinium chelates and manganese compounds, or contrast agents including perfluorocarbon compounds and biofunctionalized nanoparticles containing perfluorocarbons and gadolinium for imaging arterial plaques and atherosclerotic vessels, or SPIO and USPIO contrast agents such as ferumoxtran-10 that are used only for lymph, liver, spleen and recently brain imaging. But even as recently as Jun. 28, 2007, an update on the Magnetic Resonance—Technology Information Portal by Robert R. Edelman, Professor and Chairman, Department of Radiology at Northwestern University's Feinberg School of Medicine states that “The design objectives for the next generation of MR contrast agents will likely focus on prolonging intravascular retention, improving tissue targeting, and accessing new contrast mechanisms . . . . Technical advances in MR imaging will further increase the efficacy and necessity of tissue-specific MRI contrast agents.” (Emphasis added, see Google's cache of http://www.mr-tip.com/serv1.php?type=db1&dbs=Contrast%20Agents as retrieved on Jun. 28, 2007 21:51:08 GMT. To link to or bookmark this page, use the following url: http://www.google.com/search?q=cache:ornByMzsGVgJ:www.mr-tip.com/serv1.php%3Ftype%3Ddb1%26dbs%3DContrast%2520Agents+tissue+specific+MRI+contrast+agents&hl=en&ct=clnk&cd=2&gl=us).

### SUMMARY OF THE INVENTION

In a first embodiment of the invention there is provided a method of assessing stage of cancer in a subject, the method comprising administering a macrophage imaging agent to the subject, making a magnetic resonance image of regions of the subject's body at cancer risk, and using the image to assess macrophage density and displacement associated with any primary cancer or metastatic cancer in the subject, such density and displacement being indicative of neoplasia.

Another embodiment provides a method as described, wherein using the image includes observing macrophage activity associated with a primary tumor or with any metastatic tumor in bone, lymph node, spleen, liver, central nervous system, lung, or other organ. In particular embodiments, the regions collectively include the entire body. In other particular embodiments, the macrophage imaging agent is an ultrasmall superparamagnetic iron oxide particle and in still more particular embodiments, the macrophage imaging agent has a blood half-life sufficient to permit microphage trapping throughout the regions at cancer risk. In related particular embodiments the macrophage imaging agent is a complex of ultrasmall superparamagnetic iron oxide and a polysaccharide. In still other related embodiments, the polysaccharide is selected from the group consisting of dextran, reduced dextran and a derivative thereof.

Another particular embodiment provides a method of assessing efficacy of an anticancer treatment in a subject comprising administering a macrophage imaging agent to the subject before the anticancer treatment, making a magnetic resonance image of regions of the subject's body to be targeted by the anti-cancer treatment to establish a pre-treatment image, administering the anticancer treatment to the subject, administering the macrophage imaging agent to the subject after the anti-cancer treatment, making a magnetic resonance image of the regions of the subject's body targeted by the anticancer treatment to establish a post-treatment image, and assessing any change in the post-treatment image compared to the pre-treatment image with respect to macrophage density and displacement associated with a primary cancer or metastatic cancer in the subject, wherein assessment of such change in macrophage density and displacement is indicative of the efficacy of the anti-cancer treatment. In more particular embodiments, the anticancer treatment may be attempted extirpation or in situ ablation, chemotherapy, radiation therapy, or a combination of any of the individual treatment modalities. In still more particular embodiments, the macrophage density and displacement associated with a primary cancer or metastatic cancer is reduced or shows no change in the post-treatment image compared to the pre-treatment image. In a related embodiment, the macrophage density and displacement associated with a primary cancer or metastatic cancer is increased in the post-treatment image compared to the pre-treatment image, suggesting progression. In yet a more particular embodiment, the macrophage density and displacement associated with a primary cancer or metastatic cancer shows regression or is progression free in the post-treatment image compared to the pre-treatment image.

Another particular embodiment provides a method of determining frequency of follow-up MEMRI evaluation in a subject, the method comprising performing a first whole body MEMRI evaluation of the subject at date one to determine a first level of macrophage density at a tumor site of interest, performing a second whole body MEMRI evaluation of the subject at date two to determine a second level of macrophage density at the tumor site of interest, and determining a date three for performing a third whole body MEMRI evaluation of the subject, thereby determining the frequency of follow-up MEMRI evaluation in the subject at the tumor site of interest.

Still another particular embodiment provides a method for determining metastatic potential of cancer foci in a subject, the method comprising using whole body MEMRI evaluation to identify macrophage density at a tumor site of interest, the macrophage density at the tumor site of interest being an indicator of metastatic potential of the cancer foci and assessing the macrophage density at the tumor site of interest, thereby determining metastatic potential for the cancer foci in the subject based on the macrophage density.

Still another embodiment provides a method for determining prognosis of cancer in a subject, the method comprising performing a whole body MEMRI evaluation of the subject to identify macrophage density at a tumor site of interest, assessing the macrophage density to identify primary and/or metastatic tumors in the subject, and determining the prognosis of the cancer in the subject based on macrophage density of the primary and/or metastatic tumors, the macrophage density being an indicator of the prognosis of the cancer whereby low macrophage density relative to normal cells is an indicator of a more favorably prognosis and high macrophage density relative to normal cells is an indicator of a less favorable prognosis.

In yet another particular embodiment there is provided a report card for follow-up assessment of cancer status, the report card comprising fillable space for patient information; fillable space for date information; fillable space for initial MEMRI information; fillable space for follow-up MEMRI evaluation information; fillable space for next scheduled MEMRI evaluation; optionally, fillable space for initial diagnosis; optionally, fillable space for initial stage information; optionally; and optionally, fillable space for TNM Stage.

Still another particular embodiment provides a method for directing site of biopsy in a subject, the method comprising performing a whole body MEMRI evaluation of the subject to identify macrophage density at a tumor site of interest and assessing the macrophage density to identify the site of biopsy in the subject, macrophage density being an indicator of tumor growth.

Another particular embodiment provides a method for providing individualized cancer treatment to a subject in need thereof using whole body MEMRI evaluation, the method comprising performing a whole body MEMRI evaluation of the subject to identify macrophage density at a primary and/or tumor site of interest, assessing the macrophage density to identify characteristics (type, location, phenotypic and morphological) of the primary and/or metastatic tumors in the subject, assessing the characteristics of the primary and/or metastatic tumors in the subject to determine optimal treatment, and providing individualized cancer treatment to the subject based on the assessment of the primary and/or metastatic tumors in the subject, as determined using whole body MEMRI evaluation.

And in still another particular embodiment there is provided a macrophage biomarker capable of being administered to a subject from between 12 and 168 hours prior to whole body MEMRI evaluation.

### BRIEF DESCRIPTION OF THE DRAWINGS

The foregoing features of the invention will be more readily understood by reference to the following detailed description, taken with reference to the accompanying drawings, in which:

FIG. 1 shows an illustration of a patient placed within a whole-body MRI system for scanning, here used with a currently-approved contrast agents to visualize the arterial vessels throughout the body. (see Nael et al. (2007) Am. J. Radiol, 188, 529-39.

FIG. 2A-C show three variations of a cancer report card that may be used when practicing embodiments of the present invention.

FIG. 3 is an illustration showing a tumor region with increased macrophage density, and the process whereby tumor-associated macrophages produce chemotactic factors (CC-Chemokines, e.g. CCL2), macrophage colony stimulatory factor (M-CSF) and vascular endothelial growth factor (VEGF) to generate new blood vessels and facilitate further growth of the tumor (angiogenesis) (illustration from Allavena et al., (2006) Eur. J. Cancer, 42, 717-727).

FIGS. 4A and 4B show a patient with breast cancer with macrophages around the primary tumor and displaced from the metastatic tumor in an adjacent lymph node tumor. FIG. 4A is an in vivo MRI of the patient's breast with a contrast agent of the invention; FIG. 4B is an in vitro MRI of the removed specimen containing the tumor and a metastatic lymph node, also with a contrast agent of the invention. The arrow in FIG. 4A shows the very clear presence of a dark accumulation of macrophages indicating a tumor. FIG. 4B shows the lymph node tumor indicated by a dark outline of macrophages where the center area of the tumor is light, because the cancer cells have displaced the macrophages from this central region of the tumor.

FIGS. 5A and 5B show a patient with bladder cancer with macrophages around the primary tumors. The bladder is indicated generally with an arrow in FIGS. 5A and 5B as the large central light area in the center of the pelvis region. FIG. 5A is the MRI without contrast agent and FIG. 5B is the image with contrast agent of the invention. In FIG. 5A, the tumor's presence is only hinted at by the “crease” in the bladder (shown with an arrow) that seems to be an indication of pressure on or displacement of the bladder along this juncture. FIG. 5B, with contrast agent, clearly shows the line of demarcation for the tumor along that “crease”, with the massive tumor showing as a dark mass directly to the left of this line, continuing down to a point and back again. A second smaller tumor is indicated with an arrow to the right of the bladder, appearing as a bulls eye type node. This second tumor is outlined with a dark ring of macrophages. The center of the tumor shows up lighter where the cancer cells have displaced the macrophages. As an indication of the power of this contrast agent in cancer diagnosis and staging, this tumor is not identifiable as a tumor at all in the image (5A) with no contrast agent.

FIGS. 6A, 6B and 6C show MRI depictions of a patient with prostate cancer. The prostate is indicated generally as the large central circular space in the center of the pelvis region. FIG. 6A is the MRI without contrast agent, and FIGS. 6B and 6C are the MRIs with a contrast agent of the invention. The presence of a tumor is not indicated at all in FIG. 6A, the MRI without contrast agent. In stark comparison, FIGS. 6B and 6C indicate the presence of a very large tumor, and possibly multiple large tumors, within the prostate, as indicated by the three arrows pointing out regions of the tumor (or tumors) that are particularly enhanced with macrophage, in the presence of contrast agent. In FIG. 6C, one can more clearly see the large size of the tumor, as well as its amorphous nature (indicated by an arrow to the central left portion of the prostate), where macrophage have infiltrated the tumor and cause the tumor to appear mottled dark and light grey in this image. The presence of the macrophages provides important information about the aggressive nature of this prostate cancer.

### DETAILED DESCRIPTION OF SPECIFIC EMBODIMENTS

Definitions. As used in this description and the accompanying claims, the following terms shall have the meanings indicated, unless the context otherwise requires:

As used herein, the terms “cancer” and “cancerous” refer to or describe the physiological condition in mammals in which a population of cells are characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, melanoma and various types of head and neck cancer.

“Tumor” and “neoplasm” as used herein refer to any mass of tissue that result from excessive cell growth or proliferation, either benign (noncancerous) or malignant (cancerous) including pre-cancerous lesions.

“Metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

As used herein, the term “subject” refers to any animal (e.g., a mammal), including, but not limited to humans, non-human primates, rodents, and the like, which is to be the recipient of a particular treatment. Typically, the terms “subject” and “patient” may be used interchangeably herein in reference to a human subject.

The terms “cancer cell”, “tumor cell” and grammatical equivalents refer to the total population of cells derived from a tumor including both non-tumorigenic cells, which comprise the bulk of the tumor cell population, and tumorigenic cells.

As used herein, “assessing stage of cancer” or “staging cancer” refers to any MRI information that is useful in determining whether a patient has a primary cancer or tumor, and/or metastatic cancer or tumor, and/or information that is useful in classifying the stage of the cancer into a phenotypic category or any category having significance with regards to the prognosis of or likely response to anticancer treatment (either anticancer treatment in general or any particular anticancer treatment) of the primary or metastatic tumor(s). Similarly, assessing stage of cancer refers to providing any type of information, including, but not limited to, whether a subject is likely to have a condition (such as a tumor), and information related to the nature or classification of a tumor as for example a high risk tumor or a low risk tumor, information related to prognosis and/or information useful in selecting an appropriate treatment. Selection of treatment can include the choice of a particular chemotherapeutic agent or other treatment modality such as surgery or radiation or a choice about whether to withhold or deliver therapy.

As used herein, the terms “providing a prognosis”, “prognostic information”, or “predictive information” refer to providing information regarding the impact of the presence of cancer (e.g., as determined by the staging methods of the present invention) on a subject's future health (e.g., expected morbidity or mortality, the likelihood of getting cancer, and the risk of metastasis).

MRI. Nuclear Magnetic Resonance (NMR) Imaging, or Magnetic Resonance Imaging (MRI) as it is commonly known, is a non-invasive imaging modality that can produce high resolution, high contrast images of the interior of the human body. Magnetic resonance imaging (MRI) has proven useful in the diagnosis of many diseases such as hepatic steatosis, cancer, multiple sclerosis, sports related injury, and bone marrow disorders. MRI provides unique imaging capabilities which are not attainable in any other imaging method. For example, MRI can provide detailed images of soft tissues, abnormal tissues such as tumors, and other structures which cannot be readily imaged using techniques like X-rays. Further, MRI operates without exposing patients to ionizing radiation experienced in X-rays. For these and other reasons, MRI is commonly utilized in the medical field.

MRI involves the interrogation of the nuclear magnetic moments of a subject placed in a strong magnetic field with radio frequency (RF) magnetic fields. An MRI system typically comprises a fixed magnet to create the main strong magnetic field, a gradient coil assembly to permit spatial encoding of signal information, a variety of RF resonators or RF coils as they are commonly known, to transmit RF energy to, and receive signals emanating back from the subject being imaged, and a computer to control overall MRI system operation and create images from the signal information obtained. The large majority of RF coils used in MR imaging are tuned to ¹H due to the high abundance of this paramagnetic nucleus in the body, and the resulting ability to produce detailed structural images of body and tissue structure, although MRI using other nuclei (¹³C, ³¹P, ²³Na, ¹⁹F) is also possible.

Whole body magnetic resonance imaging (MRI) technology has been known and used for a number years. For example, U.S. Pat. No. 6,963,768 to V. B. Ho and T. K. F. Foo issued Nov. 8, 2005 (Whole body MRI scanning with moving table and interactive control), U.S. Pat. No. 6,681,132 issued Jan. 20, 2004 to J. Katz et al. (Sodium magnetic reasonance imaging used in diagnosing tumors and assessing response to treatment), U.S. Pat. No. 6,975,113 issued on Dec. 13, 2006 to D. Gurr (Method and system for moving table MRI with partial Fourier imaging) and U.S. Pat. No. 7,227,359 issued Jun. 5, 2007 to J. Ma (Method and apparatus for phase-sensitive magnetic resonance imaging) all describe various methods and systems that can be used for performing continuous whole body MRI. Similarly, US Publication No. 20050171423 to V. B. Ho and T. K. F. Foo published Aug. 4, 2005 (Whole body MRI scanning with moving table and interactive control) and US Publication No. 20050154291 to L. Zhao et al. published Jul. 14, 2005 (Method of using a small MRI scanner) also disclose whole body MRI methods and apparatus. It is envisioned that any one or more of the above-disclosed methodologies and apparatus may be useful to carry out various embodiments of the presently claimed invention. With that in mind, the entire contents of the above-referenced U.S. patents (U.S. Pat. Nos. 6,963,786; 6,681,132; 6,975,113; 7,227,359) and US Published Applications (20050171423 and 20050154291) are hereby incorporated by reference herein in their entirety.

The major hardware that comprises an MRI system includes the magnet, cryogenic systems, gradient coils, RF coils, patient table, the various amplifiers and image acquisition and processing subsystems. A whole body scanner typically requires a large enough magnet opening to accommodate whole body scans with sufficient magnetic field homogeneity, RF field homogeneity and enough RF power over large volumes to generate sufficient excitation, sufficient gradient linearity over a large volume, strength and slew rate to generate images of acceptable clarity and quality to make diagnosis of diseased organs and tissues. These in turn depend on the magnetic field strength and patient opening which determine to a large extent the overall system design, power consumption and demand on the complexity of the electronics and image acquisition and processing systems.

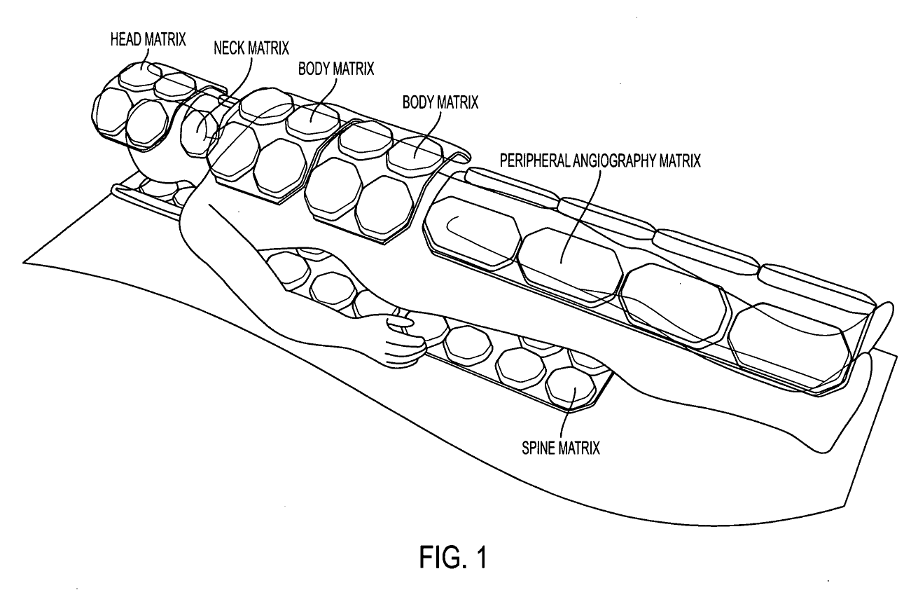

Traditional magnet systems for MRI scanners have to accommodate the insertion of a human being and generate a homogeneous region large enough to cover a cylindrical area with a diameter between about 20 to about 50 cm, preferably about 40 cm, spherical volume (DSV) over the subject. For sufficient image quality, the magnets are typically made from permanent magnets in low-field systems (<5,000 gauss; <0.5 T) and superconducting magnet systems in high field systems (>10,000 gauss; >IT). FIG. 1 shows an illustration of a patient placed within a whole-body MRI system for scanning with the use of contrast agents (see Nael et al. (2007) Am. J. Radiol, 188, 529-39).

MRI Contrast Agents. Magnetic Resonance Imaging (MRI) uses NMR (nuclear magnetic resonance) to visualize internal features of a living subject, and is useful to produce for prognosis, diagnosis, treatment, and surgery. Generally, the differences related to relaxation time constants T1 and T2 of water protons in different environments are used to generate an image. However, these differences can be insufficient to provide sharp high resolution images with adequate depiction of health or disease.

The differences in the relaxation time constants can be enhanced by contrast agents. Examples of such contrast agents include a number of magnetic agents paramagnetic agents (which primarily alter T1) and ferromagnetic or superparamagnetic (which disproportionately alter T2 response). Chelates (e.g., EDTA, DTPA and NTA chelates) can be used to attach (and reduce toxicity) of some paramagnetic substances (e.g., Fe⁺³, Mn⁺², Gd⁺³). Other agents can be in the form of particles, e.g., less than 10 μm to about 10 nM in diameter). Particles can have ferromagnetic, antiferromagnetic or superparamagnetic properties. Particles can include, e.g., magnetite (Fe₃O₄), gamma-Fe₂O₃, ferrites, and other magnetic mineral compounds of transition elements. Magnetic particles may include: one or more magnetic crystals with and without nonmagnetic material. The nonmagnetic material can include synthetic or natural polymers (such as sepharose, dextran, dextrin, starch and the like.

Embodiments of the present invention provide methods for staging, diagnosing, characterizing, and assessing cancer progression, growth and potential for and/or actual metastasis using MRI and a contrast agent. Some MRI contrast agents that may be useful in carrying out the presently claimed invention are summarized in EP0502814B1, the contents of which are hereby incorporated by reference herein.

For all cancers, staging requires information on the status of the primary tumor, the regional lymph nodes, and the evaluation of possible metastatic sites. At each of these locations, usually evaluated using the TNM system as described for breast cancer above, the activity of local macrophages provides diagnostic information. In primary tumors or metastatic sites, increased macrophage density identifies a local region of concern. In addition, the displacement of normal macrophages from lymph nodes, liver, or spleen, when appropriate to the primary tumor, identifies potential metastasis. In particular embodiments of the present invention methods of staging cancer involves whole body MRI using a macrophage-seeking contrast agent.

With the feasibility of whole body MRI scanning and the availability of an MR biomarker that accumulates in local macrophages, it becomes feasible to conduct whole body TNM staging in a single examination. One particularly useful class of MR biomarkers providing this utility are iron oxide nanoparticles. An important attribute facilitating their utility is a long blood life so that better macrophage accumulation is achieved. Two such agents are ferumoxytol and ferumoxtran-10, contrast agents that are particularly suited for use in embodiments of the presently claimed invention. Ferumoxytol and ferumoxtran-10 are MRI agents that are superparamagnetic, and fall within a class known as ultrasmall superparamagnetic particles iron oxide particles (USPIOs). In one study, useful iron oxide nanoparticles such as ferumoxtran-10 were studied for their effect on macrophages in vitro and found to be non-toxic to human monocyte-macrophages (see Gillard et al., Biomaterials 28 (2007) 1629-1642). In general, USPIOs that comprise polyols, polyethers and/or polysaccharides, particularly reduced polysaccharides, more particularly carboxyalkylated reduced polysaccharides are useful for embodiments of the whole body MRI scanning described here. In a particular embodiment, the polysaccharide of the USPIO is a carboxyalkylated reduced dextran iron oxide complex.

More particularly, MRI agents useful for embodiments of the presently claimed invention will be macrophage-seeking agents, such as the USPIOs disclosed in the following patents and applications, the contents of which are all hereby incorporated by reference herein in their entirety: U.S. Pat. No. 5,160,726 issued Nov. 3, 1992 to Josephson et al. (Filter Sterilization for Production of Colloidal Superparamagnetic MR Contrast Agents); U.S. Pat. No. 5,262,176 issued Nov. 16, 1993 to Palmacci et al. (Synthesis of Polysaccharide Covered Superparamagnetic Oxide Colloids); U.S. Pat. No. 6,599,498 issued on Jul. 29, 2003 to Groman et al. (Heat Stable Colloidal Iron Oxides Coated With Reduced Carbohydrates and Carbohydrate Derivatives); and US Publication No. 2003/0225033 A1, published Dec. 4, 2003 to Groman et al. (Heat Stable Colloidal Iron Oxides Coated With Reduced Carbohydrates and Carbohydrate Derivatives); and US Publication No. 2003/0232084 A1, published Dec. 18, 2003 to Groman et al. (Polyol and Polyether Iron Oxides Complexes Coated With Reduced Carbohydrates and Carbohydrate Derivatives). In particular embodiments the contrast agent is used as a single contrast agent. In related embodiments, the contrast agent is used in combination with another contrast agent.

Traditionally, MRI is used to evaluate tumor morphology at a single site. For example, Combidex, a monocrystalline iron oxide complex useful for practicing the present invention, has been used experimentally to evaluate metastasis to lymph nodes—visualizing the displacement of the rich macrophage population in normal nodes (see Weissleder et al., N Engl J. Med., 2003).

We have determined, surprisingly, that administration of any one of a class of macrophage-seeking contrast agents followed by a whole-body MRI enables visualization of tissue surrounded by or associated with macrophages, which tissue will be enhanced in the MR image by the macrophage-seeking contrast agent. This in turn permits staging of any solid tumor, with the identification of both primary and metastatic cancers. In addition, such MRI methods allow an assessment of anticancer therapy, by comparison of tumor number, size, morphology and location, among other characteristics, observed with MRI before treatment, between treatment cycles and after the anticancer treatment.

Using macrophage-seeking contrast agents and whole body MRI to perform a MEMRI evaluation as described above unexpectedly and surprisingly allows a physician to efficiently stage cancer for a variety of tumor types as well as assess metastasis at a much earlier point in the patient's cancer management because any tissue or organ in the entire body that has become surrounded by or associated with macrophages—a marker of the tumorigenic capabilities of that tumor—will be visualized by the whole-body MEMRI performed with any of the macrophage-seeking contrast agents described in particular embodiments of the present invention. By taking advantage of this effect in embodiments of the present invention, the physician can (a) provide a more accurate assessment of the metastatic potential of the primary tumor, (b) determine the degree of metastasis that may have already begun, (c) identify the location of the metastatic tumors, (d) customize the anticancer treatment based on the characteristics and metastatic extent of the primary tumor (or metastatic tumors already present), and (e) assess the efficacy of such treatment

Recently it has become known by those specializing in MRI that whole-body imaging is becoming more feasible and will be useful in oncology, including staging. Thus, in addition to the patents for whole body MRI described above, Paula Gould, in an “Overread” article in “Diagnostic Imaging” magazine discusses how whole-body MR imaging “should now be regarded as the test of choice for staging skeletal metastatic disease” (Whole-Body MR Imaging Outclasses Bone Scans” in Diagnostic Imaging” Apr. 1, 2007). However, the whole-body MR imaging advocated for staging skeletal metastatic disease does not propose using macrophage-seeking contrast agents to perform a MEMRI, and more importantly, misses the reason it would be advantageous to do so, not just for skeletal changes, but for the unexpected presence of macrophages. In fact, the article continues to stress that positron emission topography (PET)/computer tomography (CT), i.e. PET/CT, “is currently the best option for staging soft-tissue metastatic disease.” And, although acknowledging that whole-body MRI (again, in the absence of macrophage-enhancing contrast agents) is showing promise, a noted professor of musculoskeletal radiology in Dublin is quoted as stating that, while the emergence of diffusion-weighted techniques with whole-body MRI produce a PET-like map of the molecular movements of water, “sclerotic metastases do not have increased diffusion and will be missed using this technique.” And, as recently as April 2007 Quon et al. (Radiology, 243, pp. 204-211) continue to advocate the use of integrated FDG PET/CT imaging for detection, monitoring and its positive predictive value (PPV) for patients with bone metastases, mentioning only in the last sentence that “an additional adjunctive examination (e.g. MR imaging or biopsy) may be necessary” for patients with solitary bone lesions with discordant PET and CT findings. As with other reports, the authors do not suggest MR imaging with contrast agents, and in this case, do not suggest whole body imaging, and never disclose use of macrophage biomarkers to perform a MEMRI evaluation.

Also, Ruehm et al. (JAMA, 2003, 290, pp 3199-3206) compared the strengths of whole-body fluorine 18 fluorodeoxyglucose (FDG) PET/CT and whole-body MRI for tumor staging in oncology (for a variety of malignant diseases), but do not disclose or suggest using MR contrast agents, particularly macrophage-seeking contrast agents, to stage cancer. Moreover, the authors concluded that “[r]eflecting the more precise definition of the T-stage and N-stage status, staging of malignancies was considerably more accurate when based on whole-body PET/CT imaging compared with whole-body MRI. Based on our data, FDG-PET/CT can be recommended as a first-line modality for whole-body tumor staging.” (see Ruehm et al, p. 3204, col. 1).

Nixon et al. compared MR imaging in patients with malignant brain tumors using iron oxide nanoparticles versus gadolinium agents as the contrast agents, concluding that the iron oxide contrast agent appears to enhance areas that do not enhance with gadolinium agents and may improve post-operative imaging problems associated with gadolinium. This study focused on primary tumors in the brain, and the authors report that there was a pattern of sharply delimited cells without processes that were histologically identifiable as macrophages and another pattern of stellate-shaped cells typical of reactive astrocytes, leading them to conclude that uptake in primary brain tumors of the iron oxide contrast agent studied is primarily concentrated in reactive cells in and around the brain tumor, rather than the tumor cells themselves and so could not conclude that all of the lesions imaged with the iron oxide contrast agent were actually tumors. The authors also hypothesized that changes in residual post-operative enhancement by the iron oxide contrast agent in brain lesions compared with what is observed with gadolinium contrast agents may be caused by trauma from surgery. Nothing in the study suggests that the iron oxide nanoparticles could be useful for whole body imagining and staging of cancer in general using MEMRI evaluation.

A study in the New England Journal of Medicine by Weissleder et al. (N. Engl. J. Med. 2003, 348, pp. 2491-2499) discloses the use of MRI for detection of clinically occult lymph node metastases in prostate cancer, reporting that MRI “is relatively insensitive for the detection of lymph-node metastases [but] can be improved by using different imaging agents and acquisition techniques” particularly the use of lymphotrophic superparamagnetic nanoparticles. The authors report 100% sensitivity in identifying patients with metastases using this technique, and 96% accuracy in correctly diagnosing patients that are free of lymph node metastases (see Weissleder at 2495). However, the technique is described for detecting lymph node metastases only, and nowhere do the authors suggest that this technique is generally applicable to other metastatic diseases. A follow-up study published in 2006 by Siemens Medical Solutions USA, Inc. (Harisinghani et al., 09 2006, Siemens Medical Solutions USA Inc., Order No. A9119-61365-C₁₋₄A00, MR lymphangiography-Molecular Imaging Perspective with MR) confirmed the value of MR lymphangiography using lymphotrophic superparamagnetic nanoparticles for detecting/identifying lymph node metastases, but again did not suggest the techniques as generally applicable to other metastatic diseases other than lymph node metastases.

It has also become known by those in the area of cancer that macrophages are closely associated with tumor cells and are associated with metastasis. For example, Allavena et al., in a paper about tumor-associated macrophages as potential targets of anticancer therapy, discuss that “accumulation of leukocyte subpopulations is the hallmark of several pathological conditions, including tumors, and that a major component of the leukocytes found in tumors is macrophages. (Eur. J. Cancer (2006), 42, pp. 717-727 at 717). They go on to explain that these macrophages located in and around tumors are known as tumor-associated macrophages, abbreviated as TAM, and that immunologists see the presence of TAM as evidence of a host response against the growing tumor (id.). Others (e.g. Wyckoff et al., in Cancer Res. 2007, 67, pp. 2649-2656) report that the presence of macrophages in tumors has been correlated with poor prognosis, but until their study, there was no direct observation of how macrophages were involved in metastasis (id, at 2649).

But Applicant is the first to have the insight that the macrophage-seeking properties of certain MR contrast agents can be combined with whole-body MR imaging and surprisingly permit initial staging of a wide variety of soft tissue cancers, identification of primary and metastatic tumors with MRI using a single contrast agent, permit assessment of anticancer therapy and development of individualized therapy based on the morphology of the tumors identified, identify a site for biopsy, and provide a prognosis, because of the knowledge that macrophages associate with tumors and are an indicator of poor prognosis.

Applicant is the first to understand the surprising benefit that can be obtained by performing whole body MEMRI to stage soft tissue cancers, allowing earlier, more sensitive, and more accurate evaluation of a wide variety of metastatic tumors using an MR contrast agent that accumulates in macrophages. None of the studies summarized above realized the potential for whole body MEMRI in cancer diagnosis, staging, anticancer therapy, biopsy, prognosis, and follow-up therapy. Until Applicant's surprising discovery, it was not understood that certain contrast agents, such as the lymphotrophic iron oxide nanoparticles disclosed in Weissleder at al. and the Siemens Medical Solutions USA Inc. report had all the properties required for such improved cancer evaluation using MEMRI. The prior art teaches particular contrast agents for particular tissue imaging, whole-body imaging in the absence of contrast agents to stage bone cancer, MRI with USPIOs to assess lymph nodes for cancer metastasis, and compares gadolinium contrast agents with USPIOs in brain cancer with MRI, but no where does the prior art suggest or teach that a general, non-tumor-, but macrophage-seeking contrast agent with a long half life might be used effectively with whole body MEMRI for staging, diagnosing, assessing, providing prognosis, (and more) of soft tissue primary and metastatic tumors. In fact, a study by Guerbet of France using a USPIO contrast agent—Sineram, also known as Combidex in the U.S.A.—teaches away from Applicants surprising insight. The Guerbet study reported that characterization of breast tumors using MRI after administration of the contrast agent Sineram was not useful because no enhancement shortly after Sineram administration was seen in any of the assessed breast tumors by MRI, but all were detected using a gadolinium contrast agent (unpublished study, attached as Appendix A).

However, applicants surprisingly show that the disclosed contrast agents, which have been used primarily to image macrophage displacement in lymph node, liver, spleen, can be exploited because of their general, non-tumor-specific macrophage seeking properties and long in vivo half life, to be used with whole-body MEMRI to identify macrophage enriched regions associated with cancer foci, thereby enabling the physician to stage cancer, follow metastasis, assess prognosis, and assess anticancer treatments, among other benefits.

A possible mechanism for utility of ME-MRI is described below.

Macrophage enhancement, as described in this application, is based upon the ability of the biomarker to identify anatomic regions where normal macrophage populations are numerous, such as liver, spleen, lymph node and bone marrow as well as abnormal anatomic regions where accumulations of macrophages represents a pathophysiologic process. In the best studied class of useful biomarkers, the ultrasmall superparamagnetic nanoparticles, such as ferumoxtran-10, ferumoxytol, and ferucarbotran, their size and coating create a long-lived vascular distribution following administration. This is due to the very slow transit from the vascular space in regions that is characteristic of most of the body tissues. But there are normal tissues, such as liver, spleen, lymph node and bone marrow that have high vascular permeability. Macrophage populations in these tissues have access to the ME-MRI biomarker and trap the effective agent for subsequent imaging. Some of the pathophysiological processes that are effectively imaged also have increased vascular permeability and this is facilitated by cytokines released from the local macrophages that have accumulated in the diseased tissues. The long vascular phase and limited vascular permeability sustains a vascular reservoir of biomarker for a sufficient time to allow the improved targeting of the effective MR biomarker to the macrophages in regions of high permeability for ME-MRI.

### Example 1

### MEMRI Evaluation of Patient with Suspected Cancer in Single Breast

This situation involves a patient presenting with known or suspected cancer of one breast but a normal mammogram of the opposite breast. Recently, it has been suggested that such patients should undergo contrast-enhanced breast MRI to rule out other cancer foci [See Lehman, et al (2007) N Eng. J Med 356, 1295-303. In such an evaluation, the contrast agent is usually a gadolinium chelate and abnormal breast tissue is expected to show a focal accumulation of gadolinium in the expanded extracellular space associated with the cancer that was not clinically or mammographically evident. Though sensitive, this procedure is fraught with false positives—the abnormal regions must be biopsied and four of five such regions will not be cancerous—and does not provide information on the possible metastasis to local lymph nodes. In the present invention, the patient at risk is administered the macrophage biomarker and the breast and axilla are imaged with MRI at a time when macrophage labeling is evident—usually 12-168 hours, but preferably 24-72 hours later. Normal regional lymph nodes will accumulate the macrophage agent whereas nodal tissue replaced by metastasis will not. Most aggressive breast cancers or those with a poor prognosis show a region of accumulated excess macrophages. The presence of these macrophages is detected by the MRI examination. This detection is more specific than excessive gadolinium accumulation.

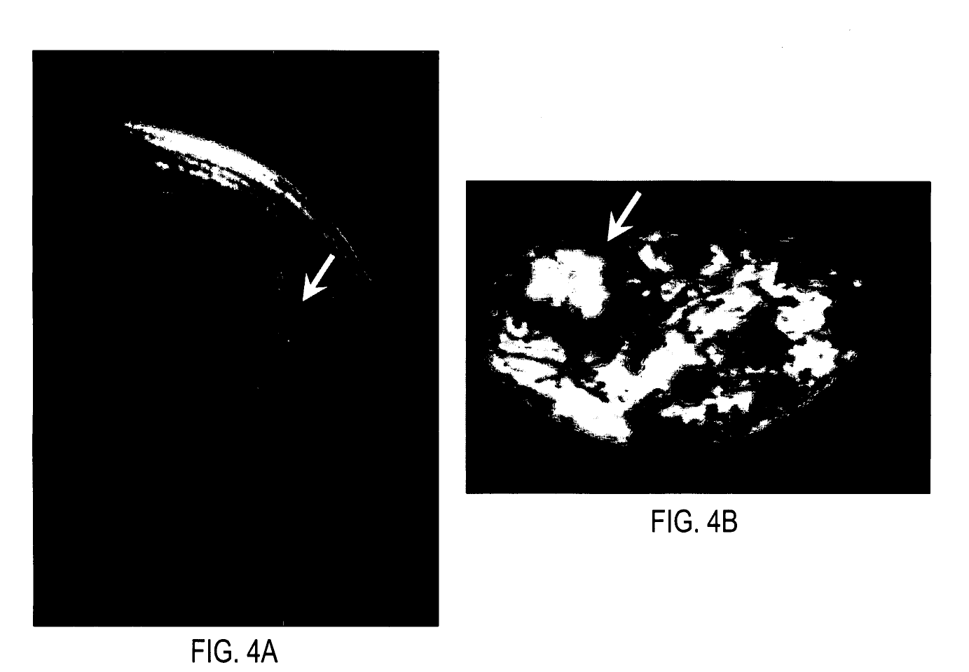

FIGS. 4A and 4B show a patient with breast cancer with macrophages around the primary tumor and displaced from the metastatic tumor in an adjacent lymph node tumor. FIG. 4A is an in vivo MRI of the patient's breast with a contrast agent of the invention; FIG. 4B is an in vitro MRI of the removed specimen containing the tumor and a metastatic lymph node, also with a contrast agent of the invention. The arrow in FIG. 4A shows the very clear presence of a dark accumulation of macrophages indicating a tumor. FIG. 4B shows the lymph node tumor indicated by a dark outline of macrophages where the center area of the tumor is light, because the cancer cells have displaced the macrophages from this central region of the tumor.

This patient was imaged following the administration of Combidex. The in vivo image in FIG. 4A identifies a primary breast tumor (arrow) and a metastatic lymph node tumor. The tissue was removed and a high resolution T2 weighted in vitro MRI performed. (FIG. 4B). With this MR pulse sequence, the macrophage enhancing agent is identified by the dark rim surrounding the primary breast cancer. Within the lymph node, normal macrophages similarly identified the lymph node tumor, but in this case there is a central zone where the macrophages have been displaced by metastatic cancer. Histopathology confirms the primary and metastatic tumors. This example shows the utility of identifying macrophages in regions where they represent pathology and the absences of macrophages from normal structures where they should be abundant.

If desired, other regions of the body can be imaged at the same time without an additional contrast administration to evaluate the presence of cancer in, for example, brain, lung, liver, or bone.

It is evident that gadolinium enhancement and macrophage enhancement can also be combined. Where desirable, the MEMRI exam is done as above, followed immediately or later by the gadolinium-enhanced MRI.

### Example 2

### Patient with Breast Cancer and Bone Pain

When metastatic breast cancer is suspected, it is important to rule out the most common sites of metastasis, as well as recurrence or new cancer in the breast. If whole body MRI is performed during macrophage enhancement, in other words, if a whole body MEMRI is performed, identification of soft tissues where there is excessive macrophage density will identify where metastasis may be present. In addition, bone MRI is the best examination for bone metastasis, and the identification of macrophage dense areas by MEMRI would increase diagnostic accuracy in bone. Finally, displacement of normal macrophages in liver, spleen, or lymph nodes would suggest the presence of metastasis in these sites. One additional use of the macrophage imaging technique MEMRI is to identify regions where a tissue biopsy may be obtained for pathological information.

### Example 3

### Patient with Bladder Cancer and/or Bone Pain

This example is similar to the above examples with breast cancer, with the additional advantage that regional nodes can be reexamined along with liver and lung, or other sites of potential metastasis.

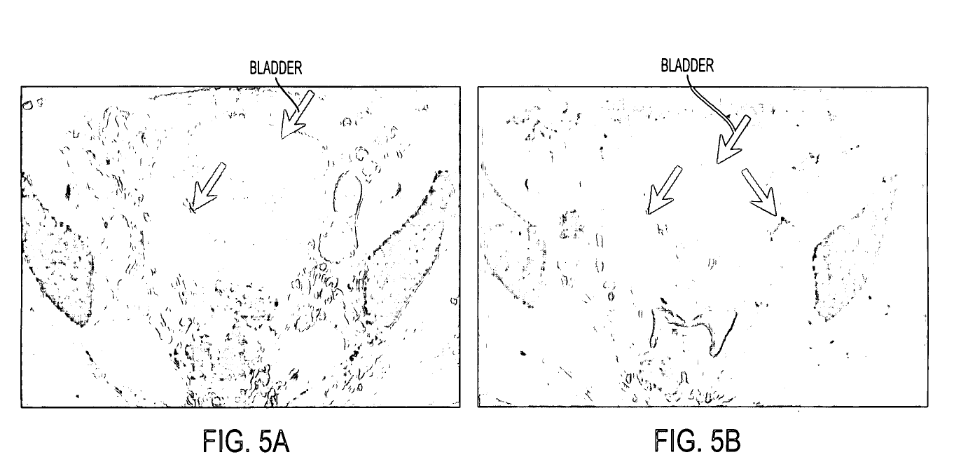

FIGS. 5A and 5B show a patient with bladder cancer with macrophages around the primary tumors. The bladder is indicated generally with an arrow in FIGS. 5A and 5B as the large central light area in the center of the pelvis region. FIG. 5A is the MRI without contrast agent and FIG. 5B is the image with contrast agent of the invention. In FIG. 5A, the tumor's presence is only hinted at by the “crease” in the bladder (shown with an arrow) that seems to be an indication of pressure on or displacement of the bladder along this juncture. FIG. 5B, with contrast agent, clearly shows the line of demarcation for the tumor along that “crease”, with the massive tumor showing as a dark mass directly to the left of this line, continuing down to a point and back again. A second smaller tumor is indicated with an arrow to the right of the bladder, appearing as a bulls eye type node. This second tumor is outlined with a dark ring of macrophages. The center of the tumor shows up lighter where the cancer cells have displaced the macrophages. As an indication of the power of this contrast agent in cancer diagnosis and staging, MRI prior to MEMRI (FIG. 5A) merely hints at a large lesion adjacent to the bladder. Following MEMRI (FIG. 5B) the lesion is seen to be large with a substantial content of macrophages and invasion of the bladder wall. The macrophage content suggests a high degree of angiogenicity and likely aggressive local tumor growth.

Once diagnosis of the primary tumor is made, whole body MRI is appropriate. If a whole body MEMRI is performed, identification of soft tissues where there is excessive macrophage density will identify where metastasis may be present. In addition, bone MRI is the best examination for bone metastasis, and the identification of macrophage dense areas by MEMRI would increase diagnostic accuracy in bone. Finally, displacement of normal macrophages in liver, spleen, or lymph nodes would suggest the presence of metastasis in these sites. One additional use of the macrophage imaging technique MEMRI is to identify regions where a tissue biopsy may be obtained for pathological information.

### Example 4

### Patient with Aggressive Prostate Cancer and Possible Bone Pain

This example is similar to the above examples with breast cancer, with the additional advantage that regional nodes can be reexamined along with liver and lung, or other sites of potential metastasis.

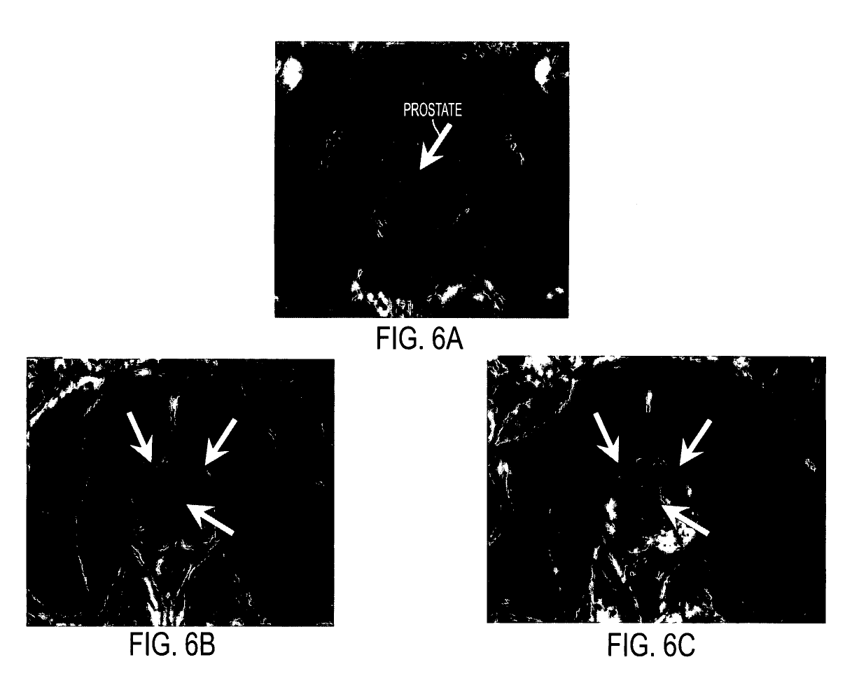

FIGS. 6A, 6B and 6C show MRI depictions of a patient with prostate cancer. The prostate is indicated generally as the large central circular space in the center of the pelvis region. FIG. 6A is the MRI without contrast agent, and FIGS. 6B and 6C are the MRIs with a contrast agent of the invention. The presence of a tumor is not indicated at all in FIG. 6A, the MRI without contrast agent. In stark comparison, FIGS. 6B and 6C indicate the presence of a very large tumor, and possibly multiple large tumors, within the prostate, as indicated by the three arrows pointing out regions of the tumor (or tumors) that are particularly enhanced with macrophage, in the presence of a contrast agent of the invention. In FIG. 6C, one can more clearly see the seemingly massive size of the tumor, as well as its amorphous nature (indicated by an arrow to the central left portion of the prostate), where macrophage have infiltrated the tumor and cause the tumor to appear mottled dark and light grey in this image.

The MRI prior to MEMRI (FIG. 6A) shows an enlarged prostate gland with little cellular discrimination. Following MEMRI (FIGS. 6B and 6C), the prostate cancer is seen to include multiple zones with surrounding macrophages. This finding is believed to reflect poor prognosis.

Once diagnosis of the primary tumor is made, whole body MRI is appropriate. If a whole body MEMRI is performed, identification of soft tissues where there is excessive macrophage density will identify where metastasis may be present. In addition, bone MRI is the best examination for bone metastasis, and the identification of macrophage dense areas by MEMRI would increase diagnostic accuracy in bone. Finally, displacement of normal macrophages in liver, spleen, or lymph nodes would suggest the presence of metastasis in these sites. Again, an additional use of the macrophage imaging technique MEMRI is to identify regions where a tissue biopsy may be obtained for pathological information.

### Example 5

### Patient with Metastatic Disease Expressing Excess Macrophage Density Undergoing Treatment

Prior to the initiation of chemotherapy with expected dose-related side effects, the sites of metastases are determined with delayed macrophage-enhanced MRI (MEMRI). As chemotherapy progresses, the reduction in macrophage density indicates efficacy whereas the unabated presence of the same or increased macrophage density indicates incomplete therapeutic response.

### Example 6

### General Whole Body MEMRI Protocol

Either T1-weighted and fast-spin echo T2-weighted images, complimented with gradient-recalled-echo (GRE) T2*-weighted sequences, or T2 and T2*-weighted sequences, are examples of imaging methods that are used with a suitable USPIO. Depending on the particular USPIO chosen, however, T1-weighted sequences alone may be sufficient. To capture a primary tumor and possible associated metastatic tumors, high resolution images are essential. Therefore, preferably, acquired images have a resolution of at least about 1-3 mm isotropic ideally, with at least 2-5 mm through plane, nominally. Siemens Medical Solutions and TIM (total imaging matrix) technology is one example system that may be utilized to acquire such high resolution images with a suitable USPIO such as ferumoxtran-10. Other useful imaging systems include the PoleStar N-10 system (Odin Medical Technologies, Yokneam Elit, Israel), the Magnetom Vision system (Siemens), the Sonata System (Siemens) using a rolling table platform (Body SURF, MR Innovation, Essen, Germany) and the Horizon system (GE Medical Systems).

Because macrophage seeking biomarkers such as ferumoxtran-10 slowly escape from the blood vessels after administration over the course of 12 to 168 hours or more, they leak into the interstitial space. They encounter monocytes that have been recruited through cytokine signals to the tumor and have been differentiated into macrophages. It is there that the macrophages will internalize the biomarker, enabling imaging of these TAMs.

T2- or T2*-weighted images appear as dark images for benign lymph, liver and spleen tumors because of the biomarker uptake by the macrophages, whereas malignant tumors of the lymph, liver and spleen appear as brighter regions on the images due to lack of uptake of the particles by the tumor cells. Such images are referred to as displacement images, and the process is sometimes also referred to by us as negative MEMRI evaluation because the normal cells are displaced by the tumor and only the normal cells are directly imaged by the USPIO biomarkers.

By contrast, the T2- or T2*-weighted images for malignant tumors in other tissues will be identifiable by a dark band of TAMs which have accumulated the USPIO.

First, non-contrast enhanced T1-weighted and T2-weighted sequences may be taken using, for example, a section width of about 7 mm. Repetition times and echo times are, for example, 124 ms and 1.8 ms, respectively, for the T1-weighted sequences, and 1200 ms and 60 ms, respectively, for the T2-weighted sequences. Subsequently, the USPIO is administered to the patient, and after approximately 12-168 h, 5-10 successive, contrast-enhanced 3-dimensional data sets are acquired as the patient is moved through the imaging cavity. With certain advances, the whole-body MRI can be acquired continuously.

It is also possible to perform MEMRI evaluations of isolated, or partial regions of the body, such as of the torso, the legs, or excluding the head and neck, etc., as needed or indicated, and as instructed by the physician.

### 7. Staging Cancer and Providing a Prognosis Using MEMRI

A patient identified as having a malignant tumor (such as by clinical exam, other imaging or biopsy) is administered a macrophage biomarker and the suspected primary is tumor imaged with MEMRI, as described in Example 5, at a time when macrophage labeling is evident—usually 12-168 hours, but preferably 24-72 hours later. TAMs will accumulate the macrophage-seeking biomarker agent in tumor sites and so these sites will thus be visible by MEMRI. Depending on the morphology of the primary tumor and the presence, number and morphology of metastatic tumors, a physician can stage the cancer. The presence of TAMs indicates sites of tumor growth, and the density of the TAMs at the sites of tumor is an indication of tumor prognosis. Aggressive cancers and/or those with a poor prognosis show a region of accumulated excess macrophages. The presence of these macrophages is detected by the MRI examination and provides an indication of tumor stage and prognosis.

Multiple regions of the body can be imaged in this way, without administration of an additional contrast agent at the same time, to evaluate the presence of cancer in tissues such as, for example, breast, brain, lung, liver, muscle or bone.

Gadolinium enhancement and macrophage enhancement can also be combined, when desirable, in which case the MEMRI exam is done as above, followed immediately or later by the gadolinium-enhanced MRI. These same techniques can be used to identify and assess the metastatic potential of cancer foci.

### 8. Determination of Individualized Anticancer Therapy and Assessment of Anticancer Therapy Using MEMRI Evaluation

A patient identified as having a malignant tumor (such as by biopsy) is administered a macrophage biomarker and the patient is imaged with MEMRI, as described in Example 5, at a time when macrophage labeling is evident—usually 12-168 hours, but preferably 24-72 hours later. TAMs will accumulate the macrophage-seeking biomarker agent at tumor sites and these sites will thus be visible by MEMRI. Depending on the morphology of the primary tumor and the presence, number and morphology of metastatic tumors, a physician can determine the aggressiveness of the cancer and whether it has a good or a poor prognosis, based on the density of TAMs visible with the initial MEMRI, with tumors having a poor prognosis exhibiting a region of accumulated excess macrophages. The presence of these macrophages is detected by the MRI examination and provides an indication of tumor stage and prognosis. This, in turn, is used to determine an individualized anticancer therapy for that patient, which may comprise chemotherapy, radiation therapy, surgery, and immunotherapy, in various combinations, sequences, or alone.

Then, at predetermined times during and after the anticancer therapy, follow-up MEMRI evaluations may be directed performed, as directed by the physician, and compared to the base-line MEMRI evaluation performed prior to the anticancer therapy. A decrease in the number of tumors or the size of the tumors, as evidenced by the observance of TAMs using MEMRI, or evidence that the macrophage density and displacement associated with a primary cancer or metastatic cancer shows regression or is progression free in the post-treatment image compared to the pre-treatment image is evidence of the efficacy of the anticancer therapy. In contrast, evidence that the number of tumors or the size of the tumors is increasing, based on the observance of TAMs using MEMRI, or evidence that the macrophage density and displacement associated with a primary or metastatic tumor is still progressing, post-treatment, instructs the physician to modify or suspend the current anticancer therapy in favor of an alternate/additional treatment regimen.

As described above, multiple regions of the body can be imaged in this way, without administration of an additional contrast agent at the same time, to evaluate the presence of cancer in tissues such as, for example, breast, brain, lung, liver, muscle or bone. In addition, as described above, gadolinium enhancement and macrophage enhancement can also be combined, when desirable, in which case the MEMRI exam is done as above, followed immediately or later by the gadolinium-enhanced MRI to establish baseline scans, after which the anticancer therapy is administered, and follow-up MEMRI and gadolinium-enhanced MRI is again performed and compared to the pre-treatment images.

These same techniques may be used to determine the frequency of follow-up MEMRI evaluations in a subject, to assess ongoing treatment, determine whether the patient is in remission, determined whether a secondary cancer has emerged in a patient, and/or look for metastasis, among other things.

### 9. Use of MEMRI to Determine a Site for Biopsy

A patient identified as being at risk for a malignant tumor because of physical indicators and evidence, such as observation of a strange mole, lump, pain, or other indicators, is administered a macrophage biomarker and the patient is imaged with MEMRI, as described in Example 5, at a time when macrophage labeling is evident—usually 12-168 hours, but preferably 24-72 hours later. TAMs will accumulate the macrophage-seeking biomarker agent at tumor sites and thus be visible by MEMRI. Depending on the morphology of the observed tumors and the presence, number and morphology of possible metastatic tumors, a physician determines a site for biopsy. Potential biopsy sites will be chosen, for example, if there is evidence of one tumor being the primary tumor. Such evidence may include an accumulation of TAMs at one suspected tumor site over another, as observed by MEMRI evaluation. Other evidence may be the morphology, size and position of a suspected tumor, as observed by MEMRI. Since the presence of TAMs indicates sites of tumor growth, the density of the TAMs at a given site will enable a physician to determine a site for biopsy. Aggressive cancers and/or those with a poor prognosis show a region of accumulated excess macrophages. In addition to providing a physician to determine a site for biopsy, the presence of tumor-associated macrophages is detected by the MRI examination through the use of macrophase-seeking biomarkers, and so MEMRI evaluation also provides an indication of tumor stage and prognosis, once the biopsy confirms that the tumor is malignant. The biopsy so obtained from a region with active TAMs may be analyzed for genetic or compositional information that may inform therapy.

Multiple regions of the body can be imaged in this way, without administration of an additional contrast agent at the same time, to evaluate the presence of cancer in tissues such as, for example, breast, brain, lung, liver, muscle or bone. Gadolinium enhancement and macrophage enhancement can also be combined, when desirable, in which case the MEMRI exam is done as above, followed immediately or later by the gadolinium-enhanced MRI. These same techniques can be used to identify and assess the metastatic potential of cancer foci.

### Example 10

### Use of a Report Card to Follow Up Treatment

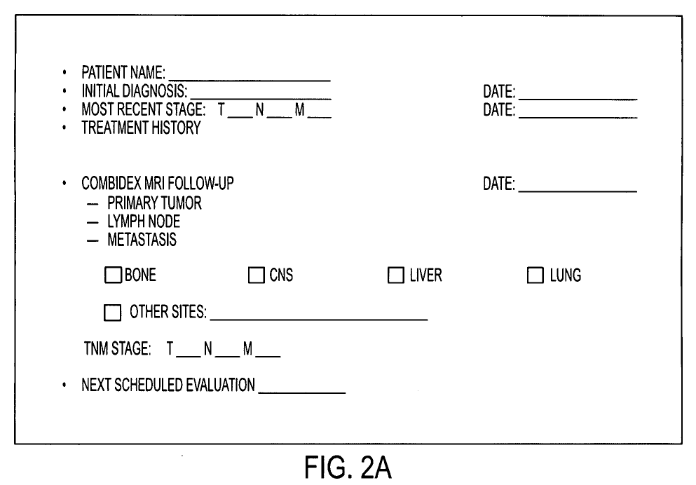

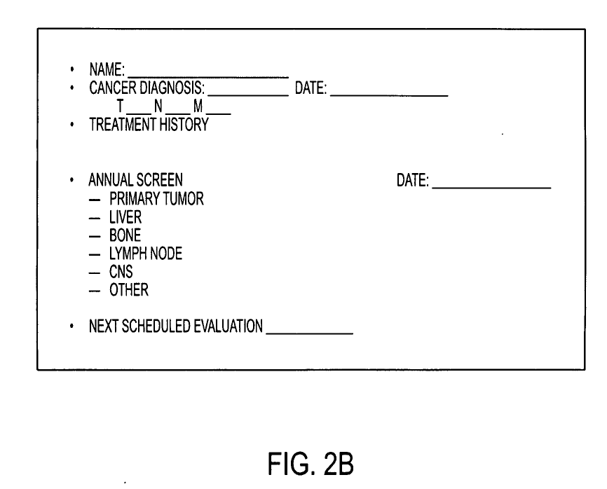

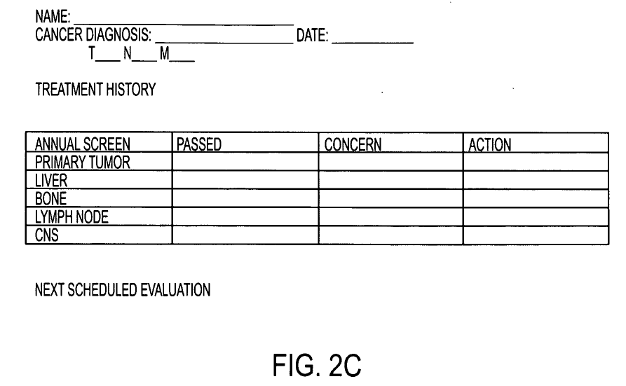

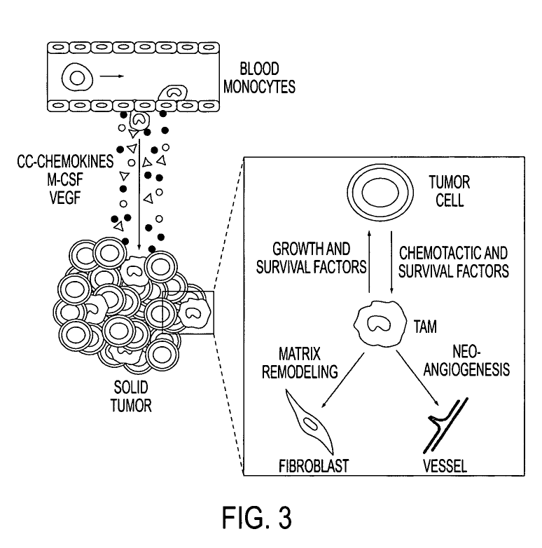

As an aid to a physician in assessing and following treatment for a given cancer patient, a report card, such as shown in FIGS. 2A, 2B and 2C made be used. The report card may include fillable spaces for patient information, the date original and new information is entered, spaces for information and date regarding an initial MEMRI, and for the next scheduled MEMRI evaluation and all follow-on MEMRI evaluations. A report card may also contain fillable spaces for information relating to the initial diagnosis, information relating to the initial stage or staging of the cancer, for information relating to the nature or cell type of the primary tumor, additional space for information relating to secondary tumors found or suspected, space for adding information relating to follow-up MEMRI evaluation information, and fillable space for information relating to standard TNM Stage procedures. Of course, any combination of categories are envisioned for such a physician report card, as well as additional categories for the report card, depending on the patient, the nature and type of cancer, and the needs of the physician. For example, the report card may be organized and/or designed to aid primarily the patient. In such an embodiment, the report card serves to provide information and a succinct summary or snapshot of the ongoing progress of the patient's disease and treatment plan, prognosis, in layperson's terms and designed to provide information that will be helpful and informative from the patient's point of view. Other embodiments may be organized and/or designed to aid primarily the physician and healthcare providers, providing a similar succinct summary or snapshot of the ongoing progress of the patient's disease and treatment plan, prognosis, but presented more technically and clinically, i.e. such a report card would be designed to include information helpful and informative from a physician's or other healthcare provider's point of view.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A method of assessing stage of cancer in a subject, the method comprising:
administering a macrophage imaging agent to the subject;
performing a magnetic resonance image of regions of the subject's body at cancer risk; and
using the image to assess macrophage density and displacement associated with any primary cancer or metastatic cancer in the subject, such density and displacement being indicative of neoplasia.

2. A method according to claim 1, wherein using the image includes observing macrophage activity associated with a primary tumor or with any metastatic tumor in bone, lymph node, spleen, liver, central nervous system, lung, or other organ.

3. A method according to any of claims 1 and 2, wherein the regions collectively include the entire body.

4. A method according to any of claims 1 through 3, wherein the macrophage imaging agent is an ultrasmall superparamagnetic iron oxide particle.

5. A method according to claim 4, wherein the macrophage imaging agent has a blood half-life sufficient to permit microphage trapping throughout the regions at cancer risk.

6. A method according to claim 5, wherein the macrophage imaging agent is a complex of ultrasmall superparamagnetic iron oxide and a polysaccharide.

7. A method according to claim 6, wherein the polysaccharide is selected from the group consisting of dextran, reduced dextran and a derivative thereof.

8. A method of assessing efficacy of an anticancer treatment in a subject, the method comprising:
administering a macrophage imaging agent to the subject before the anti-cancer treatment;
making a magnetic resonance image of regions of the subject's body to be targeted by the anti-cancer treatment to establish a pre-treatment image;
administering the anticancer treatment to the subject;
administering the macrophage imaging agent to the subject after the anti-cancer treatment;
making a magnetic resonance image of the regions of the subject's body targeted by the anti-cancer treatment to establish a post-treatment image; and
assessing any change in the post-treatment image compared to the pre-treatment image with respect to macrophage density and displacement associated with a primary cancer or metastatic cancer in the subject, wherein assessment of such change in macrophage density and displacement is indicative of the efficacy of the anti-cancer treatment.

9. A method of assessing efficacy of an anticancer treatment according to claim 8, wherein the anticancer treatment includes a treatment selected from the group consisting of chemotherapy, extirpation, in situ ablation, radiotherapy, immunotherapy, gene therapy and alternative therapy.

10. A method of assessing efficacy of an anticancer treatment according to claim 9 wherein the anticancer treatment is chemotherapy.

11. A method of assessing efficacy of an anticancer treatment according to claim 9, wherein the anticancer treatment is radiation therapy.

12. A method of assessing efficacy of an anticancer treatment according to claim 8, wherein the anticancer treatment is a combination therapy.

13. A method of assessing efficacy of an anticancer treatment according to claim 8, wherein the macrophage density and displacement associated with a primary cancer or metastatic cancer is reduced in the post-treatment image compared to the pre-treatment image.

14. A method of assessing efficacy of an anticancer treatment according to claim 8, wherein the macrophage density and displacement associated with a primary cancer or metastatic cancer is increased in the post-treatment image compared to the pre-treatment image.

15. A method of assessing efficacy of an anticancer treatment according to claim 8, wherein the macrophage density and displacement associated with a primary cancer or metastatic cancer shows regression or is progression free in the post-treatment image compared to the pre-treatment image.

16. A method of determining frequency of follow-up MEMRI evaluation in a subject, the method comprising:
performing a first whole body MEMRI evaluation of the subject at date one to determine a first level of macrophage density at a tumor site of interest;
performing a second whole body MEMRI evaluation of the subject at date two to determine a second level of macrophage density at the tumor site of interest; and
determining a date three for performing a third whole body MEMRI evaluation of the subject, thereby determining the frequency of follow-up MEMRI evaluation in the subject at the tumor site of interest.

17. An method for determining metastatic potential of cancer foci in a subject, the method comprising;
using whole body MEMRI evaluation to identify macrophage density at a tumor site of interest, the macrophage density at the tumor site of interest being an indicator of metastatic potential of the cancer foci; and
assessing the macrophage density at the tumor site of interest, thereby determining metastatic potential for the cancer foci in the subject based on the macrophage density.

18. A method for determining prognosis of cancer in a subject, the method comprising:
performing a whole body MEMRI evaluation of the subject to identify macrophage density at a tumor site of interest;
assessing the macrophage density to identify primary and/or metastatic tumors in the subject; and
determining the prognosis of the cancer in the subject based on macrophage density of the primary and/or metastatic tumors, the macrophage density being an indicator of the prognosis of the cancer whereby low macrophage density relative to normal cells is an indicator of a more favorably prognosis and high macrophage density relative to normal cells is an indicator of a less favorable prognosis.

19. A report card for follow-up assessment of cancer based, the report card comprising:
fillable space for patient information;
fillable space for date information;
fillable space for initial MEMRI information;
fillable space for next scheduled MEMRI evaluation;
optionally, fillable space for initial diagnosis;
optionally, fillable space for initial stage information;
optionally, fillable space for follow-up MEMRI evaluation information; and
optionally, fillable space for TNM Stage.

20. A method for directing site of biopsy in a subject, the method comprising:
performing a whole body MEMRI evaluation of the subject to identify macrophage density at a tumor site of interest; and
assessing the macrophage density to identify the site of biopsy in the subject, macrophage density being an indicator of tumor growth.

21. A method for providing individualized cancer treatment to a subject in need thereof using whole body MEMRI evaluation, the method comprising:
performing a whole body MEMRI evaluation of the subject to identify macrophage density at a primary and/or tumor site of interest;
assessing the macrophage density to identify characteristics (type, location, phenotypic and morphological) of the primary and/or metastatic tumors in the subject;
assessing the characteristics of the primary and/or metastatic tumors in the subject to determine optimal treatment; and
providing individualized cancer treatment to the subject based on the assessment of the primary and/or metastatic tumors in the subject, as determined using whole body MEMRI evaluation.

22. A macrophage biomarker capable of being administered to a subject from between 12 and 168 hours prior to whole body MEMRI evaluation.
